(19) United States
(12) Patent Application Publication
(10) Pub. No.: US 2013/0330742 A1
(43) Pub. Date: Dec. 12, 2013

(54) Assay for colon and other cancers in humans

(71) Applicant: Tommye Jordan, Knoxville, TN (US)

(72) Inventor: Tommye Jordan, Knoxville, TN (US)

(73) Assignee: EDP Biotech Corporation, Knoxville, TN (US)

(21) Appl. No.: 12/928,025

(22) Filed: Dec. 1, 2010

Related U.S. Application Data
(63) Continuation-in-part of application No. 12/589,588, filed on Oct. 26, 2009, now abandoned.

(51) Int. Cl. G01N 33/68; C07K 14/78; C07K 16/30

(52) U.S. Cl. CPC G01N 33/6887; C07K 16/3046; C07K 14/78
USPC 435/7.23; 530/387.9; 530/391.3; 435/188; 435/7.92; 436/501; 435/331; 530/324

(57) ABSTRACT

The present invention is concerned with a novel antigen associated with human tumors, including carcinomas of the colon, pancreas, and lung, as well as novel monoclonal antibodies which binds strongly to said antigen. The antibodies bind to normal human cells to a much lesser degree than to tumor cells. The antibodies find use both in diagnostic methods such as the detection of malignant cells associated with tumors and in therapeutic methods for treatment of humans with tumors. The novel antigen disclosed is a 60,000 Dalton glycoprotein found on the cell surface of human tumor cells.

### STATEMENT CLAIMING BENEFIT OF PRIOR-FILED APPLICATIONS

Pursuant to 37 CFR §1.78 applicant claims benefit of and priority date of the following prior-filed applications: U.S. Utility application Ser. No. 12/589,588 filed on Oct. 26, 2009; PCT application number PCT/US2010/002830, filed on Oct. 25, 2010. Both cited applications describe and claim materials and methods that are further described herein.

### FIELD OF THE INVENTION

The present invention relates to two novel monoclonal antibodies reactive with human carcinoma cells and to methods for production and use of such novel monoclonal antibodies, and use thereof to test for the presence of cancer in humans. More specifically, the monoclonal antibodies of this invention are reactive with a novel antigen which is associated with a variety of human tumors including carcinomas of the colon, lung, and pancreas.

The monoclonal antibodies of this invention are useful for diagnostic and therapeutic purposes. Uses include detecting elevated humoral levels of prognostic antigens, in vivo and in vitro imaging of malignant carcinomas, stimulation and programming of immune responses.

### BACKGROUND OF THE INVENTION

Carcinomas cause millions of deaths annually. Of all cancers, colorectal cancer is the second leading cause of cancer-related deaths in the U.S., while pancreatic cancer is the eleventh most common cancer and the fourth leading cause of cancer death in both men and women. Most cases of carcinomas are incurable by chemotherapy and radiation therapy unless detected and treated in the early stages of the disease. The more advanced a cancer is when it is diagnosed, the less likely it is that therapy will be effective. Therefore, despite the advances in cancer research, there remains a need for novel monoclonal antibodies useful for the early diagnosis and treatment of carcinomas of the colon, pancreas and lung.

Generally, monoclonal antibodies are used as invaluable reagents in diagnostics. In fact, they have played a major role in deciphering the functions of various bio-molecules in biosynthetic pathways. They have also become the reagents of choice for identification and characterization of tumor specific antigens and have become a valuable tool in the classification of cancer.

Once tumor-associated antigens have been purified from tissue extracts such antigens can be used to elicit production of antibodies to the antigen by injection into animals. Monoclonal antibodies can then be produced via hybridoma fusion techniques. (see, Kohler and Milstein, Nature, 256:495-97 (1975); Brown et al., J. Immunol., 127 (2):539-46 (1981); Brown et al., J. Biol. Chem., 255:4980-83 (1980); Yeh et al., Proc. Nat'l. Acad. Sci. (USA), 76 (6):2927-31 (1976); and Yeh et al., Int. J. Cancer, 29:269-75 (1982)).

Monoclonal antibodies reactive with carcinoma-associated antigens are known (see, e.g., Papsidero, Semin. Surg. Oncol., 1 (4):171-81 (1985); Schlom et al., Important Adv. Oncol., 170-92 (1985); Allum et al., Surg. Ann., 18:41-64 (1986); Houghton et al., Semin. Oncol., 13 (2):165-79 (1986); Monoclonal Antibodies in Cancer: Advances for Diagnosis and Treatment, Roth (ed.), Futura Publishing, Mt. Kisco, N.Y. (1986); and Cancer Diagnosis In Vitro Using Monoclonal Antibodies, Kupchik (ed.) Marcel Dekker, Inc., New York, (1988)).

Many of the known monoclonal antibodies are reactive with several types of human carcinomas, while a few antibodies react with carcinomas derived from specific organs of the body, e.g., lung, breast, ovary, colon, stomach or pancreas. (See, e.g., Fink et al., Prog. Clin. Pathol., 9:121-33 (1984)). For example, monoclonal antibodies reactive with glycoprotein antigens on specific types of carcinomas include those described in U.S. Pat. No. 4,737,579 (monoclonal antibodies to non-small cell lung carcinomas); U.S. Pat. No. 4,753,894 (monoclonal antibodies to human breast cancer); U.S. Pat. No. 4,579,827 (monoclonal antibodies to human gastrointestinal cancer); and U.S. Pat. No. 4,713,352 (monoclonal antibodies to human renal carcinoma).

Monoclonal antibodies reactive with glycolipid antigens that are believed to be associated with certain tumor cells have also been disclosed. For example, see Kniep et al., J. Immunol., 131 (3):1591-94 (1983) and U.S. Pat. No. 4,507,391 (monoclonal antibody to human melanoma).

In addition, monoclonal antibodies reactive with glycolipid antigens found on specific types of carcinoma cells include those described by Rosen et al., Cancer Research, 44:2052-61 (1984) (monoclonal antibodies to human small cell lung cancer); Varki et al., Cancer Research, 44:681-87 (1984) (monoclonal antibodies to human adenocarcinomas of the lung, stomach and colon and melanoma); and U.S. Pat. No. 4,579,827 (monoclonal antibodies to human colon adenocarcinoma).

Additional monoclonal antibodies exhibiting a reactivity to antigens associated with tumor cells are greatly needed.

In vitro diagnostic methods are known in the art and include immunohistological detection of tumor cells (e.g., on human tissue, cells or excised tumor specimens) or serologic detection of tumor-associated antigens (e.g., in blood samples or other biological fluids). Immunohistological techniques involve contacting a biological specimen such as a tumor tissue specimen with the antibody of the invention and then detecting the presence on the specimen of the antibody complexed to its antigen. The formation of such antibody-antigen complexes with the specimen indicates the presence of tumor cells in the tissue. Detection of the antibody on the specimen can be accomplished using techniques known in the art, such as the immunoperoxidase staining technique, the avidin-biotin (ABC) technique or immunofluorescence techniques (see Ciocca et al., Meth. Enzymol., 121:562-79 (1986); Kimball (ed.), Introduction To Immunology (2nd Ed.), pp. 113-117, Macmillan Publ. Co. (1986)).

Serologic diagnostic techniques involve the detection and quantitation of tumor-associated antigens that have been secreted or “shed” into the serum or other biological fluids of patients thought to be suffering from carcinoma. Such antigens can be detected in the body fluids using techniques known in the art such as radioimmunoassays (RIA) or enzyme-linked immunosorbent assays (ELISA) wherein an antibody reactive with the “shed” antigen is used to detect the presence of the antigen in a fluid sample (see, e.g., Uotila et al., J. Immunol. Methods, 42:11 (1981); Allum et al., “Monoclonal Antibodies in the Diagnosis and Treatment of Malignant Conditions” Surg. Ann., 18:41-64, 48-51 (1986); Sikora et al. (eds.), Monoclonal Antibodies, pp. 32-52, Blackwell Scientific Publications, (1984)).

However, prior to the invention presented herein serological techniques have not produced results robust enough to be useful for mass cancer screening. Serum markers such as CEA, CA19-9 and CA242 have all been shown to have poor sensitivity and specificity colon cancer. CEA was discovered in 1969 and believed to be a sensitive and specific marker for colon cancer. However, further studies were unable to produce the original results. It was shown that at a cutoff concentration of 2.5 ug/L, CEA screening would yield a sensitivity of 30%-40% and a specificity of 87%. Utilizing these numbers, for every 1 colon cancer patient identified with a CEA-based assay there would be 250 false-positives and 60% of cancers would be missed. Due to these overall poor results these markers are not used for colon cancer screening.

For this reason Fecal Occult Blood (FOB) tests have long been the mainstay of colon cancer screening. Large randomized studies have shown that screening with serial FOB tests reduces mortality from colon cancer. Typical examples of FOB tests come in two types, the guaiac-based tests available under the trade names SKB-Hemoccult II & Hemocult II SENSA, and the immune-chemical tests available under the trade names SKB-HemeSelect and Entrerix-InSure Fit.

Disadvantages of guaiac-based FOB tests include burdensome dietary restrictions, the inconvenient collection process, the limited single application sensitivity, and the costs associated with poor specificity (5-10% rate of false positives). FOB tests have sensitivity of 25%-40% and specificity of 80%-90%.

While the immune-chemical based FOB tests have the advantages of improved compliance due to ease of use and no diet restrictions, sensitivity issues remain.

It is thus apparent that a monoclonal antibody reactive with an antigen expressed at high levels by a variety of tumors may become useful towards an earlier diagnosis of cancers, the immunological monitoring of cancer patients, as well as for development of improved methods for therapy of cancers. It is also apparent that purified antigens associated with carcinomas derived from specific organs of the body can be of value for creating such monoclonal antibodies, as well as for creating cancer vaccines. It is also apparent that improved materials and methods for serological assays to identify the presence of cancer in humans would be valuable.

### SUMMARY OF THE INVENTION

An object of the present invention provides for monoclonal antibodies, or portions of monoclonal antibodies (peptides) having specificity directed to epitopes of human lung, colorectal, and pancreatic carcinoma-associated antigen. The present invention provides two such monoclonal antibodies, the CA11-19 antibodies, which specifically bind a particular antigen, the EDP antigen, which is associated with human tumor cells, particularly cells from lung, pancreas, and colon carcinomas. Thus, the antibodies of the invention can be useful for the diagnosis and therapy of tumors associated with the EDP antigen identified by the CA11-19 antibodies. The CA11-19 antibodies of the invention show no significant reactivity with normal human cells.

The antibodies of the present invention may be used for in-vitro diagnostic methods for determining the presence of a malignant condition in human colon tissue and other human tissues. One such method involves using the antibody, antibodies, or antibody fragments of the present invention in an ELISA assay. Another such method involves using the antibody, antibodies, or antibody fragments of the present invention for detection of tumor cells within biopsied tissue through the use of immunohistochemistry techniques.

Another object of the present invention provides in-vivo diagnostic methods for the localization of a tumor by administering to a patient a purified antibody, antibodies, or antibody fragments of the present invention labeled with an agent which gives a detectable signal. The localization is then detected using external scintigraphy, emission tomography, radionuclear scanning, or other known methods of imaging.

The invention also has therapeutic applications, since the CA11-19 antibodies and similar antibodies can react with the EDP antigen that is expressed in high concentrations at the tumor cell surface. The monoclonal antibody of the invention may be used in immunoconjugates as a carrier of various agents which have an antitumor effect, including, but not restricted to, chemotherapeutic drugs, toxins, immunological response modifiers, and radioisotopes.

Another object of the present invention provides methods for purifying EDP antigen.

### DETAILED DESCRIPTION OF INVENTION

In order that the invention herein described may be more fully understood, the following detailed description is set forth.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms “a,” “an, and she” include the plural reference unless the context clearly indicates otherwise. Thus, for example, the reference to an antibody is a reference to one or more such antibodies, including equivalents thereof known to those skilled in the art. Other than in the operating examples, or where otherwise indicated, all numbers expressing quantities of ingredients or reaction conditions used herein should be understood as modified in all instances by the term “about.” The term “about” when used in connection with percentages may mean ±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains.

For the purposes of this application the following terms shall have the following meanings:

An “epitope” refers to that portion of any molecule capable of being recognized by, and bound by, an antibody (the corresponding antibody binding region may be referred to as a paratope). In general, epitopes consist of chemically active surface groupings of molecules, for example, amino acids or sugar side chains, and have specific three-dimensional structural characteristics as well as specific charge characteristics.

An “antigen” is a molecule or a portion of a molecule capable of being bound by an antibody. An antigen may have one or more than one epitope. An antigen will bind in a highly selective manner with its corresponding antibody and not with the multitude of other antibodies which may be evoked by other antigens.

An “antibody” includes both intact immunoglobulin molecules as well as portions, fragments, peptides and derivatives thereof, such as, for example, Fab, Fab′, F(ab′)2, Fv, CDR regions, or any portion or peptide sequence of the antibody that is capable of binding antigen or epitope. An antibody is said to be “capable of binding” a molecule if it is capable of specifically reacting with the molecule to thereby bind the molecule to the antibody.

Antibody also includes chimeric antibodies, anti-idiotypic (anti-Id) antibodies to antibodies that can be labeled in soluble or bound form, as well as fragments, portions, regions, peptides or derivatives thereof, provided by any known technique, such as, but not limited to, enzymatic cleavage, peptide synthesis, or recombinant techniques. Such antibodies of the present invention are capable of binding portions of EDP antigen or EDP antigen-bearing cells. Antibody fragments or portions may lack the Fc fragment of intact antibody, clear more rapidly from the circulation, and may have less non-specific tissue binding than an intact antibody. Examples of antibody may be produced from intact antibodies using methods well known in the art, for example by proteolytic cleavage with enzymes such as papain (to produce Fab fragments) or pepsin (to produce F(ab′)2 fragments). See e.g., Wahl et al., 24 J. Nucl. Med. 316-25 (1983). Portions of antibodies may be made by any of the above methods, or may be made by expressing a portion of the recombinant molecule. For example, the CDR region(s) of a recombinant antibody may be isolated and subcloned into the appropriate expression vector. See, e.g., U.S. Pat. No. 6,680,053.

The present invention concerns the novel monoclonal antibodies, designated the CA11-19 antibodies, which are specifically reactive with an antigen (EDP antigen) associated with human tumor cells, particularly from carcinomas of the lung, pancreas, and colon, methods for producing the CA11-19 monoclonal antibodies, and diagnostic and therapeutic methods employing the antibodies. The CA11-19 antibodies react with a range of tumors while showing essentially no reactivity with normal human tissues or other types of tumors such as melanomas or lymphomas.

The monoclonal antibodies of the present invention can be prepared by hybridoma fusion techniques. (see, Kohler and Milstein, Nature, 256:495-97 (1975); Brown et al., J. Immunol., 127 (2):539-46 (1981); Brown et al., J. Biol. Chem., 255:4980-83 (1980); Yeh et al., Proc. Nat'l. Acad. Sci. (USA), 76 (6):2927-31 (1976); and Yeh et al., Int. J. Cancer, 29:269-75 (1982)). These techniques involve the injection of an immunogen (e.g., purified antigen or cells or cellular extracts carrying the antigen) into an animal (e.g., a mouse) so as to elicit a desired immune response (i.e., production of antibodies) in that animal. In Example I a preparation from human carcinoma of the colon designated is used as the immunogen. The tissue preparation is injected, for example, into a mouse, and after a sufficient time the mouse is sacrificed and somatic antibody-producing lymphocytes are obtained. Antibody-producing cells may be derived from the lymph nodes, spleens and peripheral blood of primed animals. Spleen cells are preferred. Mouse lymphocytes give a higher percentage of stable fusions with the mouse myelomas described below. The use of rat, rabbit and frog somatic cells is also possible. The spleen cell chromosomes encoding desired immunoglobulins are immortalized by fusing the spleen cells with myeloma cells, generally in the presence of a fusing agent such as polyethylene glycol (PEG). Any of a number of myeloma cell lines may be used as a fusion partner according to standard techniques; for example, the P3-NS1/1-Ag4-1, P3-x63-Ag8.653 or Sp2/O-Ag14 myeloma lines. These myeloma lines are available from the American Type Culture Collection (ATCC), Rockville, Md.

The resulting cells, which include the desired hybridomas, are then grown in a selective medium, such as HAT medium, in which unfused parental myeloma or lymphocyte cells eventually die. Only the hybridoma cells survive and can be grown under limiting dilution conditions to obtain isolated clones. The supernatants of the hybridomas are screened for the presence of antibody of the desired specificity, e.g., by immunoassay techniques using the antigen that has been used for immunization. Positive clones can then be subcloned under limiting dilution conditions, and the monoclonal antibody produced can be isolated. Various conventional methods exist for isolation and purification of the monoclonal antibodies so as to free them from other proteins and other contaminants. Commonly used methods for purifying monoclonal antibodies include ammonium sulfate precipitation, ion exchange chromatography, and affinity chromatography (see, e.g., Zola et al., in Monoclonal Hybridoma Antibodies: Techniques and Applications, Hurell (ed.) pp. 51-52 (CRC Press 1982)). Hybridomas produced according to these methods can be propagated in vitro or in vivo (in ascites fluid) using techniques known in the art (See, generally, Fink et al., supra, at page 123, FIG. 6-1).

Generally, the individual cell line may be propagated in vitro, for example in laboratory culture vessels, and the culture medium containing high concentrations of a single specific monoclonal antibody can be harvested by decantation, filtration or centrifugation. Alternatively, the yield of monoclonal antibody can be enhanced by injecting a sample of the hybridoma into a histocompatible animal of the type used to provide the somatic and myeloma cells for the original fusion.

It should be understood that the present invention encompasses the CA11-19 antibodies described herein and any fragments thereof containing the active binding region of the antibody, such as Fab, F(ab)₂ and Fv fragments. Such fragments can be produced from the CA11-19 antibodies using techniques well established in the art (see, e.g., Rousseaux et al., in Methods Enzymol., 121:663-69, Academic Press (1986)).

In addition, the present invention encompasses antibodies that are capable of binding to the same antigenic determinant as the CA11-19 antibodies and competing with the CA11-19 antibody for binding at that site. These include antibodies having the same antigenic specificity as the CA11-19 antibodies but differing in species origin, isotype, binding affinity or biological functions (e.g., cytotoxicity). For example, class, isotype and other variants of the antibody of the invention may be constructed using recombinant class-switching and fusion techniques known in the art (see, e.g., Thammana et al., Eur. J. Immunol., 13:614 (1983); Spira et al., J. Immunol. Meth., 74:307-15 (1984); Neuberger et al., Nature, 312:604-08 (1984); and Oi et al., supra)). Thus, chimeric antibodies or other recombinant antibodies (e.g., antibody fused to a second protein such as a lymphokine) having the same binding specificity as the CA11-19 antibodies fall within the scope of this invention. Furthermore, since the EDP antigen to which the antibodies of the present invention binds is a novel tumor antigen, the antibodies of the invention includes antibodies that bind to any antigenic determinant on that EDP antigen, including determinants other than that with which the CA11-19 antibodies react.

Also included within the scope of the invention are anti-idiotypic antibodies of the CA11-19 antibodies. These anti-idiotypic antibodies can be produced using the CA11-19 antibodies as immunogen and are useful for diagnostic purposes in detecting humoral response to tumors and in therapeutic applications, e.g., in a vaccine, to induce an anti-tumor response in patients (See, e.g., Nepom et al., Cancer And Metastasis Reviews, 6:487-501 (1987); and Lee et al., Proc. Nat'l. Acad. Sci. (USA), 82:6286-90 (1985)).

For certain therapeutic applications chimeric (mouse-human) or human monoclonal antibodies may be preferable to murine antibodies because patients treated with mouse antibodies generate human antimouse antibodies. (Shawler et al., J. Immunol., 135:1530-35 (1985)). Human monoclonal antibodies may be made by using the EDP antigen of the invention, to sensitize human lymphocytes to the antigen in vitro followed by EBV-transformation or hybridization of the antigen-sensitized lymphocytes with mouse or human lymphocytes as described by Borrebaeck et al. (Proc. Nat'l. Acad. Sci. (USA), 85:3995-99 (1988)). Therefore, human monoclonal antibodies or chimeric antibodies that bind EDP antigen are also included within the scope of the present invention.

According to one embodiment, the antibodies of this invention, designated CA11-19, were produced via hybridoma techniques using tissue from colon carcinoma as the immunogen. The CA11-19 hybridomas, producing the CA11-19 antibodies, have been deposited with the American Type Culture collection (ATCC), 12301 Parklawn Drive, Rockville, Md. 20852 under the designations 5A1-1 and 5E5-1. Accession numbers will be provided upon identification by ATCC.

The CA11-19 antibodies display very strong reactivity with tumor cells, particularly cells from colon, pancreas, and lung carcinomas. The antibodies of this invention do not display any immunohistologically detectable binding to normal human tissues such as fibroblasts, endothelial cells, or epithelial cells from the major organs.

The monoclonal antibodies of the invention are useful for diagnostic applications, both in vitro and in vivo, for the detection of human carcinomas carrying the EDP antigen with which the CA11-19 antibody is specifically reactive. In vitro diagnostic methods include immunohistological detection of tumor cells (e.g., on human tissue, cells or excised tumor specimens) and serologic detection of tumor-associated antigens (e.g., in blood samples or other biological fluids).

Immunohistological techniques involve contacting a biological specimen such as a tumor tissue specimen with the antibodies of the invention and then detecting the presence on the specimen of the antibodies complexed to their antigen. The formation of such antibody-antigen complexes with the specimen indicates the presence of tumor cells in the tissue. Detection of the antibodies on the specimen can be accomplished using techniques known in the art, such as the immunoperoxidase staining technique, the avidin-biotin (ABC) technique or immunofluorescence techniques (see, e.g., Ciocca et al., Meth. Enzymol., 121:562-79 (1986); Hellstrom et al., Cancer Research, 46:3917-23 (1986); and Kimball (ed.), Introduction To Immunology (2nd Ed.), pp. 113-117, Macmillan Publ. Co. (1986)).

Serologic diagnostic techniques involve the detection and quantitation of tumor-associated antigens that have been secreted or “shed” into the serum or other biological fluids of patients thought to be suffering from carcinoma. Such antigens can be detected in the body fluids using techniques known in the art such as radioimmunoassays (RIA) or enzyme-linked immunosorbent assays (ELISA) wherein an antibody reactive with the “shed” antigen is used to detect the presence of the antigen in a fluid sample (see, e.g., Uotila et al., J. Immunol. Methods, 42:11 (1981) and Allum et al., “Monoclonal Antibodies in the Diagnosis and Treatment of Malignant Conditions” Surg. Ann., 18:41-64, 48-51 (1986)). These assays, using the CA11-19 antibodies disclosed herein, therefore can be used for the detection in biological fluids of the EDP antigen with which the CA11-19 antibodies react and thus the detection of various carcinomas in human patients. Thus, it is apparent from the foregoing that the CA11-19 antibodies of the invention can be used in most assays involving antigen-antibody reactions. These assays include, but are not limited to, standard RIA techniques, both liquid and solid phase, as well as ELISA assays, immunofluorescence techniques, and other immunocytochemical assays (see, e.g., Sikora et al. (eds.), Monoclonal Antibodies, pp. 32-52, Blackwell Scientific Publications, (1984)).

The CA11-19 antibodies of the invention are also useful for in vivo diagnostic applications for the detection of human tumors. One such approach involves the detection of tumors in vivo by tumor imaging techniques using the antibodies labeled with an appropriate imaging reagent that produces detectable signal. Imaging reagents and procedures for labeling antibodies with such reagents are well known (see, e.g., Wensel and Meares, Radio Immunoimaging and Radioimmunotherapy, Esevier, N.Y. (1983); Colcher et al., Meth. Enzymol., 121:802-16 (1986)). The labeled antibodies may be detected by a technique such as radionuclear scanning (see, e.g., Bradwell et al. in Monoclonal Antibodies for Cancer Detection and Therapy, Baldwin et al. (eds.), pp. 65-85, Academic Press (1985)).

The CA11-19 antibodies of the invention have a number of in vivo therapeutic applications. In addition to being used alone to target tumor cells, the antibodies can be used in conjunction with an appropriate therapeutic agent to treat human cancer. For example, the antibodies can be conjugated or linked to a therapeutic drug or toxin for delivery of the therapeutic agent to the site of the cancer. Techniques for conjugating such therapeutic agents to antibodies are well known (see, e.g., Amon et al., Monoclonal Antibodies And Cancer Therapy, Reisfeld et al. (eds.), pp. 243-56, Alan R. Liss, Inc., (1985); Hellstrom et al. in Controlled Drug Delivery (2nd ed.), Robinson et al. (eds.), pp. 623-53, Marcel Dekker, Inc., (1987); Thorpe, Monoclonal Antibodies '84: Biological And Clinical Applications, Pinchera et al. (eds.), pp. 475-506 (1985); and Thorpe et al., Immunol. Rev., 62:119-58 (1982)).

Alternatively, the antibodies can be coupled to a source of high-energy radiation, e.g., a radioisotope such as ¹³¹I, which, when localized at the tumor site, results in a killing of several cell diameters (See, e.g., Order, in Monoclonal Antibodies For Cancer Detection And Therapy, Baldwin et al. (eds.), pp. 303-16, Academic Press, (1985)).

Furthermore, chimeric or other recombinant CA11-19 antibodies of the invention, as described earlier, may be used therapeutically. For example, a fusion protein comprising at least the antigen-binding region of the CA11-19 antibodies joined to at least a functionally active portion of a second protein having anti-tumor activity, e.g., a lymphokine or oncostatin, may be used to treat human tumors in vivo. In addition, a chimeric CA11-19 antibody wherein the antigen-binding region of CA11-19 is joined to a human Fc region, e.g., IgG1, may be used to promote antibody-dependent cellular cytotoxicity or complement mediated cytotoxicity.

It is apparent, therefore, that the present invention encompasses pharmaceutical compositions, combinations and methods for treating human tumors. For example, the invention includes pharmaceutical compositions for use in the treatment of human tumors comprising a pharmaceutically effective amount of a CA11-19 antibodies and a pharmaceutically acceptable carrier. The compositions may contain the CA11-19 antibodies, either unmodified, or conjugated to a therapeutic agent (e.g., drug, toxin, enzyme or second antibody). The compositions may additionally include other antibodies or conjugates for treating carcinomas (e.g., an antibody cocktail).

The antibody compositions of the invention can be administered using conventional modes of administration, including, but not limited to, intravenous, intraperitoneal, oral, intralymphatic or administration directly into the tumor. Intravenous administration is preferred.

The antibody compositions of the invention may be in a variety of dosage forms which include, but are not limited to, liquid solutions or suspensions, tablets, pills, powders, suppositories, polymeric microcapsules or microvesicles, liposomes, and injectable or infusible solutions. The preferred form depends upon the mode of administration and the therapeutic application.

The antibody compositions also preferably include conventional pharmaceutically acceptable carriers and adjuvants known in the art such as human serum albumin, ion exchangers, alumina, lecithin, buffer substances such as phosphates, glycine, sorbic acid, potassium sorbate, and salts or electrolytes such as protamine sulfate.

The present invention also concerns the novel antigen, referred to as EDP antigen. EDP antigen can be used purified via the methods set forth below. EDP antigen may be used for therapeutic applications. For example, the purified EDP antigen may be administered alone as an immunogen or together with a proper immunological adjuvant.

In order that the invention described herein may be more fully understood, the following examples are set forth. It should be understood that these examples are for illustrative purposes only and are not to be construed as limiting the scope of this invention in any manner.

### EXAMPLE I

Isolation and Characterization of EDP Antigen:

Tumor carcinomas of the gastrointestinal tract were minced, homogenized and extracted with 1.4M perchloric acid for 60 minutes and then centrifuged at 10,000 rpm for 30 minutes. The extract supernatant was extensively dialyzed against water, lyophlized and reconstituted to suitable concentrations with PBS pH 7.5. Protein band MW˜60,000 daltons was isolated by preparative PAGE electrophoresis (7.5% acrylamide gels) and further absorbed of contaminating glycoproteins by CNBr-Sepharose 48 affinity chromatography using anti-whole normal serum; ′anti-aI-acid glycoprotein, anti-01-antitrypsin, anti-albumin and anti-CEA, proteins which were found to co-extract with perchloric acid treatment.

Antigen purified in this manner was then injected into rabbits. After a suitable antibody titer was developed in the rabbits further isolation began with the perchloric acid extraction described above. Supernatant was then purified via affinity chromatography using the anti-EDP antigen antibodies obtained from the rabbits. At this point the affinity columns against other proteins, described above, were used again. Antigen isolated by both methods demonstrate single band purity by SDS-PAGE analysis.

EDP antigen has a molecular weight of about 60,000 Daltons with a secondary band at 36,000 Daltons when subjected to polyacrylamide gel electrophoresis. Thin layer isoelectric focusing reveals that EDP antigen has a pI of 4.5 in polyamylamide gels with a pH gradient from 3 to 8. EDP antigen has a peak UV absorbance at 230 nm wavelength.

EDP antigen was labeled with iodine-125 using the Iodogen method (Pierce Biochemicals). Radiolabeled EDP antigen showed a single band on sliced gel eluates obtained from SDS-PAGE electrophoresis of the labeled antigen.

The N-terminus sequence of EDP antigen is: SEQ ID NO: 1. While sequencing of the N-terminus of EDP antigen was clear for several residues, some residues in the sequence yielded results leaving uncertainty as to one out of two possible amino acids for a given residue. SEQ ID NO: 2 represents the N-terminus amino acid sequence of EDP antigen with all minor yield amino acids replacing the major yield amino acids. Some combination of SEQ ID NO: 1 and SEQ ID NO: 2 represents the The N-terminus amino acid sequence of EDP antigen.

### EXAMPLE II

Preparation of the CA11-19 Monoclonal Antibody:

The CA11-19 monoclonal antibody of the invention was produced using hybridoma fusion techniques described previously.

Preferred materials and methods for thawing, maintaining, and freezing of the 5A1-1 and 5E5-1 hybridoma cell lines are as follows:

Materials used are listed in Table 1, below.

TABLE 1

| Item | Supplier | Cat # | Internal Part # |
|---|---|---|---|
| DMEM (Dulbecco's Modified; | Invitrogen | 11965-804 | 3276 |
| High Glucose) | | | |
| Penicillin-Streptomycin Solution | Invitrogen | 15070-063 | 3306B |
| Fetal Calf Serum | Invitrogen | 26140-079 | 3278 |
| Fetal Calf Serum-Low Ig* | Invitrogen | 16250-078 | NEW |
| Sodium Pyruvate | Sigma | S8636 | 3279 |
| Glutathionine | Sigma | G6013 | 3280 |
| HT Media Supplement | Sigma | H0137 | 3288 |
| 2-Mercaptoethanol | Sigma | M7522 | 3635 |
| DMSO (dimethyl sulfonyloxide) | Sigma | D2650 | 3357 |

Preparation of Growth Medium: 1000 ml DMEM, 100 ml Fetal Calf Serum (must be heat inactivated at 56 degrees C. for 45 minutes, 10 ml 2 MEQ (MEQ is used to reconstitute HT), 2 vials HT(50×), 10 ml Sodium Pyruvate, and 30 ml Pen Strep were mixed.

Preparation of 100× 2 MEQ: 40 microliters 2-mercaptoethanol, and 10 mg Glutathione q.s. to 100 ml in Sterile Tissue Culture water were mixed.

Thawing of frozen cells: Cells were quick thawed in 37° C. water bath by shaking vial rapidly in water until approximately ¾ thawed, with a small pea-sized portion still frozen. Cells were removed from water bath, but shaking was continue to vial until it has thawed completely. The vial was rinsed with ethanol and opened carefully, pre-warmed growth medium was pipetted drop-wise to the vial with continuously flicking of vial to mix (1 drop from pasture pipette every 30 seconds) until vial is almost full. Cells were seed into a 24-well plate and a small flask T25 with appropriate volume of pre-warmed growth medium. Each well in the 24-well plate contained 1 to 2 ml of the pre-warmed media prior to seeding. The T25 flask contained at least 5 ml of the pre-warmed media prior to seeding. The flask and plate were placed in a 37° C. CO2 water-jacketed incubator. During the first 10 days cells were checked every other day for the attachment of cells using an inverted microscope. In 3 to 10 days the medium was changed if the majority of growing cells had attached to the plate or flask. Cells were split as required to maintain proper cell density.

Freezing cells: Freezing Medium was mixed by adding 10 ml of Fetal Calf Serum to 0.5 ml DMSO (Sigma D2650) and sterile filtering into a sterile 15 ml screw cap centrifuge tube. Cells were actively growing for a good recovery. Cells were fed with fresh pre-warmed growth medium the day before freezing. All Freezing Medium and cryovials were kept in ice bath during the freezing procedure. Cells were dispersed by vigorously hitting the flask against the palm of the hand. Cells were centrifuge in a refrigerated centrifuge for 5 minutes at 1500 rpm. The supernatant was removed from the cell pellet using sterile technique. 10 ml fresh DMEM was added to the cell pellet and the cells were mixed by gently tapping. Cells were centrifuge again in a refrigerated centrifuge for 5 minutes at 1500 rpm. Again, the supernatant was removed from the cell pellet. 1.5 ml fresh Freezing Medium was added to cell pellet and mix cells by gently tapping. Cells were mixed well and placed in ice bath. 20 microliters of cell mixture was removed and cell count was determined using 20 microliters 0.2 trypan blue mix to determine viability. Using the cell count the final volume of Freezing Medium to be added to the cells was determined and added to the cell suspension. Contents of the tube was placed in appropriately labeled cryovials (1.0 ml/vial) and was placed in ice bath. The cryovials with cells were placed in freezing rack of cryofreezer and frozen at a rate of 1 degree per 20 minutes. The vials were transferred to liquid nitrogen storage container reached the vials reached −130° C.

### EXAMPLE III

Immunohistochemical Staining of Tissue Samples:

Tissue sections derived from Adenocarcinoma of the colon, normal colon tissue adjacent to the neoplastic site, fetal liver and fetal lung sections were all examined using direct immunofluorescent techniques for the presence of EDP antigen and for the presence of CEA.

The antibodies (Monoclonal and Polyclonal) to Tennessee Antigen and CEA (DAKO, Denmark) were separately labeled with fluorescein isothiocyanate.

Examination of the sections by means of fluorescence microscopy demonstrated that the primary staining of the malignant tissue was associated mainly with cell plasma membranes. No staining above background was evident in sections of adult lung tissue. The staining seen in the malignant tissue using fluorescently-tagged anti CEA serum was similar to the pattern seen with anti EDP antigen stained with CA11-19 antibodies.

Cross-inhibition studies demonstrated that unlabeled anti-CEA serum did not inhibit staining for EDP antigen by fluorescently-tagged anti EDP antigen serum. Also, unlabeled anti-EDP antigen serum did not inhibit the staining by the labeled anti-CEA serum.

### EXAMPLE IV

Preparation of 96-Well Assay Plates for CA11-19 ELISA Assay:

Reagents and Materials: Microtiter Plate-Flat Bottom (Part No. 3590) Catalogue No. 2588 Costar; Elisa Coating Buffer (Product No. EDP (JCL) 5-014-B); Enzyme PBS Tween 20 (Product No. EDP (JCL) 5-008); Phosphate Buffered Saline (Product No. EDP (JCL) 3-003); Anti CA1-18 Monoclonal Antibody Prep (5E5-1); Anti CA1-18 Monoclonal Antibody Prep (5A1-1); 2% Sucrose, 4% Polyvinylpyrrolidone Solution (Product No. EDP (JCL) 5-015); Fish Serum (Part No. 3832).

Preparation of Coating Buffer (Product No. EDP (JCL) 5-014-B): 1.592 grams of sodium carbonate (Part No. 1080), 2.93 grams of Sodium Bicarbonate (Part No. 1071), and 0.2 grams of sodium azide (Part No. 1096) were added to 1000 ml of sterile distilled water. The solution was tested to assure a usable pH between 9.5 and 9.7.

Selection of anti-CA11-19 monoclonal antibodies and IgG preparation: An appropriate volume of monoclonal antibody 5E5-1 was determined to have a concentration between 0.006-0.009 mg/ml with an ideal concentration of 0.007 mg/ml. An appropriate volume of monoclonal antibody 5A1-1 was determined to have a concentration between 0.002-0.004 mg/ml with an ideal concentration of 0.003 mg/ml. Protein concentrations of monoclonal antibodies were determined using EDP (JCL) Procedure X-22.

Preparation of the monoclonal antibody cocktail: CA11-19 Monoclonal antibody 5E5-1 was added to Coating Buffer to a final concentration of 0.007 mg/ml. Anti-CA11-19 monoclonal antibody 5A1-1 also added to the solution containing the 5E5-1, giving a final concentration of 5A1-1 of 0.003 mg/ml in the final cocktail mixture containing both monoclonal antibodies. The solution was mixed with a magnetic stirrer for at least 30 minutes and was then filtered through a Costar 0.45 mm filter.

Attachment of the Monoclonal antibody cocktail to ELISA assay plates: 125 miroliters of the antibody cocktail was added to each well of the 96-well plates. The plates were placed in plastic ziplock bags on a vibrator for five minutes. The plates were then incubated at room temperature for two hours followed by 24 hour incubation at 4-8 degrees C. Upon completion of incubation excess monoclonal cocktail coating solution was removed by aspiration. Plates were immediately washed six times with 300 ul Enzyme PBS-Tween 20/well. Plate wells were not allowed to dry prior to the six washes. After the last wash, the Enzyme PBS-Tween 20 was removed from the plate. The plates were manually blotted on a clean absorbent lint free laboratory towel. Plates were not allowed to dry completely. Plates were rapidly blocked by adding 300 ul/well (+/−10 ul) of 1.2% Fish Serum consisting of 1.2 ml Fish Serum in 100 ml coating buffer. Plates were incubated with 1.2% Fish Serum Solution for two hours at 20-25 degrees C. Plates were kept covered to prevent evaporation during the Fish Serum incubation. Upon completion of incubation the wells were aspirated removing all Fish Serum Solution from wells. Again, the wells were not allowed to dry completely. Wells were rapidly blocked by adding 300 ul/well (+/−10 ul) of 2% Sucrose, 4% Polyvinylpyrrolidone solution. Plates were then incubated 45 minutes at 20-25° C. The PVP solution was removed from the wells, and the plates were vigorously blotted on lint free laboratory towels to dry completely. Coated/blocked microtiter plates were then placed inverted on polypropylene racks overnight at 20-25° C. in Clean Room with no air flow. Coated/Blocked Microtiter plates were air dried overnight to complete dryness following PVC Blocking. Plates were then placed in an aluminum foil zip-lock bag with a humidity indicator and a dessicant and seal thoroughly. Plates were stored at 4°-8° degrees C. until use.

### EXAMPLE V

Serum Assay for EDP Antigen:

Utilizing the CA11-19 Assay described herein the distribution of EDP antigen values in serum of patients with various malignant and non-malignant diseases, as well as in healthy subjects, was determined in over 2,422 individuals. 2000 samples were taken from asymptomatic patients. The remaining 422 samples were collected from patients with various GI symptoms and conditions confirmed by colonoscopy. 83 of the 422 samples were collected from patients with colon cancer confirmed via colonoscopy and histological documentation.

5 ml samples of human blood were collected from all subjects using standard venipuncture techniques. Serum was separated from other blood fractions using standard techniques.

25 μl of Patient Serum Specimens and 75 μl of Sample Diluting Buffer were pipetted into their assigned wells in the prepared CA11-19 Coated 96-well plates. Standards and controls were also diluted in Sample Diluting Buffer to give a total volume of 100 μl pipetted into assigned wells. Plates were sealed and incubated at 37° C.+/−2° C. incubator overnight, a minimum of 12 hours to a maximum of 24 hours. Wells were aspirated and washed three times with deionized water.

100 μl of Goat Anti-CA11-19 Alkaline Phosphastase Conjugate solution was added to each well. The plates were sealed and incubated for 2 hours at 37° C.+/−2° C. and washed three times with deionized water. 100 μl Substrate Solution was added to each well and the plates were incubated at 37° C.+/−2° C. for a minimum of 30 minutes. Following the latest incubation optical density at 410 or 405 nm was determined on an ELISA plate reader. Plates were periodically read at ten minute intervals looking for optical density reading of CA11-19 STANDARD I (20 u/ml) to be 1.00 or greater. Incubation was continued until CA1-18 STANDARD I (20 u/ml) optical density reached 1.0 or greater. 50 μl of 3N NaOH was dispensed in each well when CA1-18 STANDARD I (20 u/ml) reading reached 1.00 optical density at 410 or 405 nm wavelength. All wells were read at 410 or 405 nm wavelength.

Final concentrations for Sample Diluting Buffer were:

| A. Enzyme Phosphate Buffered Saline | |
|---|---|
| 1. Sodium Chloride | 0.8% |
| 2. Potassium Phos. (Mono) | 0.02% |
| 3. Sodium Phos. (Dibasic) | 0.115% |
| 4. Potassium Cl | 0.02% |
| 5. Sodium Azide in sterile Distilled H₂O | 0.02% |
| B. Tweene 20 | 0.05% |
| C. Bovine Serum Albumin (Fraction V) | 0.5% |
| D. Normal Rat Serum | 2-4% |

(each batch (lot number) has to be titered against panel of known serums)

Final concentrations for Sample Substrate Solution were:

| | |
|---|---|
| A. Diethanolamine | 10% |
| B. Magnesium Chloride | 0.15% |
| C. Sodium Chloride | 0.2% |

D. HCL Concentrate, to stabilize pH at 9.8

Study results reflected a mean level of EDP antigen for cancer patients at 7.99 units and a mean value for asymptomatic individuals of 4.75 units. Analysis of these results give a standard deviation of 1.257 units for the cancer patient group and a standard deviation of 1.46 units for the asymptomatic group. This reflects a clear and statistically significant shift in EDP Antigen levels for cancer patients when compared to non-cancer patients. Setting a cutoff of 6.5 units for positive cancer results this statistically significant difference allows for identification of 100% of patients with stage I through stage III colon cancer, while simultaneously eliminating 95% of the non-cancerous patients.

An earlier study using the CA1-18 EIA Assay measuring Tennessee Antigen directly in serum samples from cancer patients, active non-cancer patients and normal individuals (no evidence of disease) also supports the usefulness of the assay described herein. Results from that earlier study are as shown in Table 2.

TABLE 2
Initial Results Using CA1-18 EIA Assay measuring Tennessee Antigen
Directly in Serum Samples from Cancer Patients, Active Non-Cancer
Patients, and Normal Individuals

| Subject category | No. | % Neg | % Pos |
|---|---|---|---|
| Cancer Patients* | 33 | 18.0% | 82.0% |
| Non-Cancer Diseases | 19 | 79.0% | 21.0% |
| Normal Individuals | 26 | 92.3% | 7.7% |

*Cancer patients included those with Carcinoma of Colon, Rectum, Lung, Breast and Pancreas.

Results from yet another study are summarized in Table 3, below. Blood samples from 129 patients were taken with 56 known colon cancer patients including patents at all stages of the disease. The study included a group of healthy blood donors, some of whom were smokers. Smokers were defined as smoking at least one pack of cigarettes per day. All diagnoses listed as cancer were histologically confirmed.

The blood samples were tested using the ELISA test described above resulting in identification of 56 of the cancer positive patient's samples, 18 false negative results, and 55 correctly identified negative patient samples. When the maximum negative value is assigned to 6.5 units, the test has a negative predictive value of >99% (at a 95% confidence level).

TABLE 3

| Clinical Status | |
|---|---|
| Positive | Negative |

Assuming a negative cut off value of 6.5 units

| | | | |
|---|---|---|---|
| Test | Positive | 56 | 18 |
| | Negative | 0 | 55 |

Assuming a negative cut off value of 7.0 units

| | | | |
|---|---|---|---|
| Test | Positive | 53 | 12 |
| | Negative | 3 | 61 |

Assuming a negative cut off value of 8.0 units

| | | | |
|---|---|---|---|
| Test | Positive | 44 | 5 |
| | Negative | 12 | 68 |

It is apparent that many modifications and variations of this invention as set forth above may be made without departing from the spirit and scope. The specific embodiments described are given by way of example only, and the invention is limited only by the terms of the appended claims.

What is claimed is:

1. A monoclonal antibody produced by a hybridoma cell line, which antibody binds to a determinant site on an antigen of human tumor cells, said antigen characterized by a molecular weight of about 60,000 Daltons, as determined by polyacrylamide gel electrophoresis, and having a terminal amino acid sequence comprising SEQ ID NO: 1.

2. The monoclonal antibody of claim 1 wherein said antibody is produced by a hybridoma cell culture designated 5A1-1.

3. The monoclonal antibody of claim 1 wherein said antibody is produced by a hybridoma cell culture designated 5E5-1.

4. A method for producing an assay for the detection of EDP antigen comprising:
a) combining a first species of a monoclonal antibody that binds EDP antigen with a second species of a monoclonal antibody that also binds EDP antigen; and
b) binding said combined first and second species of monoclonal antibodies to a solid surface.

5. The method of claim 4 wherein said solid surface is an ELISA plate.

6. The method of claim 4 wherein said first species of said monoclonal antibody is produced by a hybridoma cell culture designated 5A1-1.

7. The method of claim 4 wherein combining said second species of said monoclonal antibody is produced by a hybridoma cell culture designated 5E5-1.

8. The method of claim 4 wherein a ratio of said of said monoclonal antibody to said second species of said monoclonal antibody is within a range between 1:2 to 1:3.

9. The method of claim 4 wherein a ratio of said of said monoclonal antibody to said second species of said monoclonal antibody is within a range between 2:1 to 3:1.

10. An immunoassay for the detection of EDP antigen comprising:
a) combining one or more monoclonal antibodies reactive with an antigen associated with human tumor cells, said antigen characterized by a molecular weight of about 60,000 daltons as determined by polyacrylamide gel electrophoresis and having an amino terminal amino acid sequence comprising SEQ ID NO: 1, with a human tissue or fluid sample; and
b) assaying for binding of EDP antigen to said one or more monoclonal antibodies.

11. The immunoassay of claim 10 wherein said monoclonal antibodies are the CA11-19 antibodies produced by hybridoma cell lines deposited with ATCC.

12. The immunoassay of claim 10 wherein said one or more monoclonal antibodies are a combination of an antibody produced by a hybridoma cell culture designated 5A1-1 and an antibody produced by a hybridoma cell culture designated 5E5-1.

13. The immunoassay of claim 10 wherein said monoclonal antibodies are labeled with a label selected from the group consisting of a radionuclide, an enzyme, a fluorescent agent and a chromophore.

14. The immunoassay of claim 10 wherein said binding of EDP antigen to said one or more monoclonal antibodies is detected by ELISA assay.

15. The method of claim 10 wherein said binding of EDP antigen to said one or more monoclonal antibodies is detected by adding additional antibodies capable of binding EDP antigen.

16. The method of claim 15 wherein said additional antibodies capable of binding EDP antigen are labeled with a label selected from the group consisting of a radionuclide, an enzyme, a fluorescent agent and a chromophore.

17. The method of claim 10 wherein said assaying for binding of EDP antigen to said one or more monoclonal antibodies is performed by immunohistological staining.


SEQUENCE LISTING (2 sequences)

SEQ ID NO: 1 - protein, 26 aa, Homo Sapiens
VETRTDVVFDLPFLMIIDPTDSQDIF

SEQ ID NO: 2 - protein, 25 aa, Homo Sapiens
  misc._feature (4)..(4): Xaa represents unknown amino acid, most likely Leu
LVKXGIKRFAGPFSIIVPTDSEDIF